(19) United States
(12) Patent Application Publication
(10) Pub. No.: US 2012/0289954 A1
(43) Pub. Date: Nov. 15, 2012

(54) MICRO PLASMA HEAD FOR MEDICAL APPLICATIONS

(76) Inventor: Amnon Lam, Kibuz Givat Oz (IL)

(21) Appl. No.: 13/508,946

(22) PCT Filed: Nov. 8, 2010
(86) PCT No.: PCT/IL10/00923

§ 371 Date: Aug. 3, 2012

Related U.S. Application Data
(60) Provisional application No. 61/259,353, filed on Nov. 9, 2009.

(51) Int. Cl. A61B 18/14

(52) U.S. Cl. 606/33

(57) ABSTRACT

According to the current invention a medical catheter for plasma ablation within a body cavity is provided, comprising: an elongated catheter having: an input gas lumen; an output gas lumen; and electrical energy supply cable; a gas supply system, supplying gas to the input gas lumen; a gas pump, pumping spent gas from the output gas lumen; a plasma head at distal end of said elongated catheter and situated in proximity to body part to be ablated, the plasma head comprising: an outer tube defining plasma space between the outer tube and the body part to be ablated, wherein input gas enters the plasma space through the input gas lumen, and spent gas is pumped out of the plasma space through the output gas lumen; and a plasma electrode, receiving electrical power from the electrical energy supply cable and generating plasma in the plasma space.

### FIELD OF THE INVENTION

The present invention relates to an apparatus and method for medical applications, for example treating Chronic Total Occlusion (CTO) of blood vessels using a catheter having micro plasma head at its distal end.

### BACKGROUND OF THE INVENTION

A Chronic Total Occlusions (CTO) is defined as, “an obstruction of a native coronary artery for greater than 30 days with no luminal continuity.

Percutaneous revascularization of CTOs represents an important clinical challenge in interventional cardiology. These lesions account for approximately 10% of all Percutaneous Transluminal Coronary Angioplasty (PTCA) procedures. However, the angioplasty of CTOs is frequently limited by both an inability to successfully pass a guidewire across the occlusion and the higher incidence of restenosis.

Improvements in angioplasty equipment, operator technique, and the introduction of innovative devices designed specifically for CTOs have improved the likelihood of successful recanalization.

However, revascularization of CTOs is technically difficult and remains a challenge for percutaneous coronary interventions. Crossing the complex lesion and reentering into the true lumen remains the key step that limits the overall success of percutaneous procedures. Successful recanalization has traditionally been achieved in only up to 60% of chronic occlusions using conventional techniques. The availability of new specialized guidewires designed to cross occluded arteries has only modestly improved procedural success rate. An example for such device is the LuMend Frontrunner Catheter (LuMend Inc, Redwood City, Calif.).

Selectively removing material from a substrate is known in the art and is used extensively in semiconductor processing and other manufacturing processes. For example, ions present in the plasma may interact with the substrate and enhance material removal. Generally ions in the plasma enhance a chemical process by striking the surface of the substrate, and subsequently breaking the chemical bonds of the atoms on the surface in order to make them more susceptible to reacting with the molecules of the chemical process. Since ion etching is mainly perpendicular, while the chemical etching is both perpendicular and vertical, the perpendicular etch rate tends to be much faster than in then horizontal direction. In thus plasma etching tends to have an anisotropic profile.

U.S. Pat. No. 7,186,659 titled “Plasma etching method”; to Fujimoto, Kotaro and Shimada, Takeshi; discloses an etching method for etching semiconductor devices, involves introducing etching gas in etching chamber, and exciting etching gas to plasma state to etch the material.

Applying electrically generated plasma to medical application is known in the art.

For example, electrosurgery surgery is known in the art and is performed by electrical methods. Its development has been driven by the clinical need to control bleeding during surgical procedures. While heat has been used medically to control bleeding for thousands of years, the use of electricity to produce heat in tissue has only been in general use since the mid 1920's, and in flexible endoscopy since the 1970's. Electrosurgery offers at least one unique advantage over mechanical cutting and thermal application: the ability to cut and coagulate tissue at the same time. This advantage makes it the ideal surgical tool for the gastroenterologist.

Electrosurgical Generators provide the high frequency electrical energy required to perform electrosurgery and some of these are equipped with an option to use argon gas enhanced electrosurgery. Argon gas enhanced or Argon Plasma Coagulation (APC) has been in long use in the operating room setting and is used intermittently, usually for parenchymal organ surgeries.

Argon plasma equipped electrosurgery systems were adapted to be able to be used in flexible endoscopic procedures of the gut and lung.

U.S. Pat. No. 6,197,026; titled “Electrosurgical instrument”; to Farin, Gunter and Grund, Karl Ernst; discloses an electrosurgical instrument for plasma coagulation of biological tissue e.g. for treating blood clots, haemostasis, thermal devitalization or destruction of pathological tissue.

US application 20080119843; titled “Compact electrosurgery apparatuses”; to Morris, Marcia; discloses a compact electrosurgical apparatus for use in electrosurgery such as flexible endoscopy.

U.S. Pat. No. 6,890,332; titled “Electrical discharge devices and techniques for medical procedures”; to Truckai, Csaba and Shadduck; discloses a medical instrument coupled to a source for introducing a gas to controllably form and capture transient gas volumes in a microchannel structure at the working surface of the instrument that interfaces with a targeted tissue site. Each of the microchannel features of the working surface carries an electrode element coupled to the electrical source. The energy may be applied to the targeted site in either of two modes of operation, depending in part on voltage and repetition rate of energy delivery. In one mode of energy application, electrical potential is selected to cause an intense electrical arc across the transient ionized gas volumes to cause an energy-tissue interaction characterized by tissue vaporization. In another preferred mode of energy delivery, the system applies selected levels of energy to the targeted site by means of an energetic plasma at the instrument working surface to cause molecular volatilization of surface macromolecules thus resulting in material removal. Both modes of operation limit collateral thermal damage to tissue volumes adjacent to the targeted site.

Optical emission spectroscopy is known in the art and is commonly used to identify chemical composition and abundance of chemical species in mixtures. Plasma may excite the mixture, and the emitted fluorescence is collected and analyzed in a spectrometer.

U.S. Pat. No. 5,083,004; titled “Spectroscopic plasma torch for microwave induced plasmas”; to Wells, Gregory and Bolton, Barbara; discloses spectroscopic plasma torch suitable for use at atmospheric pressure.

### SUMMARY OF THE INVENTION

The present invention relates to an apparatus and method for treating CTO and other complete or partial occlusions of blood vessels using a catheter having a micro plasma had at its distal end.

According to an exemplary embodiment of the current invention a medical catheter for plasma ablation within a body cavity is provided said catheter comprising: an elongated catheter having: an input gas lumen; an output gas lumen; and electrical energy supply cable a gas supply system, supplying gas to said input gas lumen; a gas pump, pumping spent gas from said output gas lumen; a plasma head at distal end of said elongated catheter and situated in proximity to body part to be ablated, said plasma head comprising: an outer tube defining plasma space between said outer tube and said body part to be ablated, wherein input gas enters said plasma space through said input gas lumen, and spent gas is pumped out of said plasma space through said output gas lumen; and a plasma electrode, receiving electrical power from said electrical energy supply cable and generating plasma in said plasma space.

In some embodiments, the invention may be applied to outer surface of the body

In some embodiments the input gas lumen and said output gas lumen are coaxial.

In some embodiments the input gas lumen is centrally to output gas lumen.

In some embodiments the wall of said input gas lumen is electrically conductive and is part of said electrical energy supply cable.

In some embodiments the catheter further comprises an optical spectrometer; and wherein signal from said spectrometer, indicative of type of said ablated body part, is used for controlling the ablation process.

In some embodiments the catheter further comprises an optical fiber transmitting light generated by plasma in said plasma space to said optical spectrometer, and wherein said optical spectrometer is situated outside said catheter.

In some embodiments controlling the ablation process comprises termination of said ablation.

In some embodiments controlling the ablation process comprises directing the ablation process.

In some embodiments directing the ablation process comprises steering said plasma head.

In some embodiments the spectrometer comprises a plurality of optical sensors each fitted with an optical filter having a predefined optical transmission.

In some embodiments least one of said a plurality of optical filter having a predefined optical transmission is tuned to the emission wavelength of phosphorus.

In some embodiments the catheter further comprises at least two spectrometers, wherein: each of said spectrometer comprises an array of a plurality of optical sensors, each fitted with an optical filter having a predefined optical transmission; wherein said spectrometers are facing said plasma space, and wherein comparing signals from said plurality of spectrometers is used for aiming the ablation process.

In some embodiments the spectrometer is integrated in an electro-optical chip.

In some embodiments the catheter further comprising an optical imager imaging said ablated body part.

In some embodiments the electro-optical chip define together with outer tube defining said plasma space between said outer tube and said body part to be ablated.

In some embodiments the electro-optical chip further comprises said plasma electrode.

In some embodiments the electro-optical chip further comprises electrical circuit, optionally including RF impedance matching network, wherein said electrical condition electrical energy and transmits said conditioned electrical energy to said plasma electrode.

In some embodiments the outer tube further comprises grounding electrodes on its inner surface, wherein said grounding electrodes is used for return plasma current from said plasma electrode.

In some embodiments the outer tube further comprises grounding electrodes on its outer surface, wherein said grounding electrodes is used for return plasma current from said plasma electrode.

In some embodiments, electric return line is fitted with variable impedance device such as a variable resistor or resistor network, allowing to change the plasma production nature between mono-polar and bi-polar and combination of the like.

In some embodiments the outer tube further comprises a grounding electrode pad attached to the patient body, wherein said grounding electrode pad is used for return plasma current from said plasma electrode.

In some embodiments the gas supply system supplies gas comprising gases selected from a group of: argon; helium; oxygen and SF6.

According to another exemplary embodiment of the current invention method for plasma ablation within a body cavity is provided comprising the steps of: supplying to a plasma head at the distal end an elongated catheter: input gas through an input gas lumen within said elongated catheter; electrical energy through electrical energy supply cable within said elongated catheter; generating plasma in said input gas using said electrical energy, wherein said plasma ablating parts of the body cavity; and pumping spent gas including ablation products through an output gas lumen within said elongated catheter.

In some embodiments the invention may be applied to outer surface of the body.

In some embodiments the input gas lumen and said output gas lumen are coaxial.

In some embodiments the method further comprises the steps of: spectrally analyzing light emitted as result of said plasma; and using said spectral analysis for controlling the ablation process.

In some embodiments the method further comprises the steps of: spatially analyzing light emitted as result of said plasma; and using said spatial analysis for aiming the ablation process.

According to an exemplary embodiment of the current invention a medical catheter for plasma ablation within a body cavity is provided said catheter comprising: an elongated catheter having: an input gas lumen; an output gas lumen; and electrical energy supply cable; a gas supply system, supplying gas to said input gas lumen; a gas pump, pumping spent gas from said output gas lumen; a plasma head at distal end of said elongated catheter and situated in proximity to body part to be ablated, said plasma head comprising: an outer tube defining plasma space between said outer tube and said body part to be ablated, wherein input gas enters said plasma space through said input gas lumen, and spent gas is pumped out of said plasma space through said output gas lumen; and a plasma generating coil, receiving electrical power from said electrical energy supply cable and inductively generating plasma in said plasma space.

It is another aspect of the current invention to provide a method for plasma production and within a body cavity comprising the steps of: supplying to a plasma head within a body cavity, input gas through an input gas lumen; providing RF power to at least a first and a second electrodes; and producing bi-polar plasma.

In some embodiments the RF power comprises a high frequency signal modulated at low frequency.

In some embodiments the method further grounding the body and producing plasma which is a combination of bi-polar and mono-polar plasma.

In some embodiments the method further comprises actively controlling the production of said plasma.

In some embodiments controlling the plasma production comprises measuring emission spectra of said plasma.

In some embodiments measuring emission spectra comprises transmitting radiation emitted by said plasma through a wall of a gas tube.

In some embodiments controlling plasma production comprises measuring reflected RF power.

In some embodiments controlling plasma production comprises measuring RF impedance.

In some embodiments temperature of the plasma is less than 80 degrees Celsius.

It is another aspect of the current invention to provide a medical device for plasma treatment capable of producing both mono-polar and bi-polar plasma.

It is another aspect of the current invention to provide a medical device for plasma treatment capable of inductively producing plasma.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 schematically depicts a block diagram of a plasma system for treating CTO according to an exemplary embodiment of the current invention.

FIG. 2 schematically depicts the distal end of a catheter of a plasma system for treating CTO inserted in a totally occluded blood vessel according to an exemplary embodiment of the current invention.

FIG. 3a schematically depicts distal end of a catheter showing some details of a bi-polar plasma head for CTO ablation according to an exemplary embodiment of the current invention.

FIG. 3b schematically depicts distal end of a catheter showing some details of a mono-polar plasma head for CTO ablation according to an exemplary embodiment of the current invention.

FIG. 3c schematically depicts a cross section of a plasma distal end of a catheter showing some details of inductively generated plasma head for CTO ablation according to another exemplary embodiment of the current invention.

FIG. 3d(i) schematically depicts a cross section of a dual purpose plasma head in bi-polar configuration, according to another exemplary embodiment of the current invention.

FIG. 3d(ii) schematically depicts a cross section of a dual purpose plasma head in mono-polar ablation or coagulation configuration, according to another exemplary embodiment of the current invention.

FIG. 4 schematically depicts a base plate having integrated sensors according to another aspect of the current invention.

FIG. 5a schematically depicts the electrical connections of a bi-polar plasma system according to an exemplary embodiment of the current invention.

FIG. 5b schematically depicts the electrical connections of a mono-polar plasma system according to an exemplary embodiment of the current invention.

### DESCRIPTION OF THE PREFERRED EMBODIMENTS

The present invention relates to an apparatus and method for treating CTO and other complete or partial occlusions of blood vessels using a catheter having a micro plasma had at its distal end.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

In discussion of the various figures described herein below, like numbers refer to like parts.

The drawings are generally not to scale. Some optional parts were drawn using dashed lines.

For clarity, non-essential elements were omitted from some of the drawings.

As used herein, an element or step recited in the singular and proceeded with the word “a” or “an” should be understood as not excluding plural elements or steps, unless such exclusion is explicitly recited.

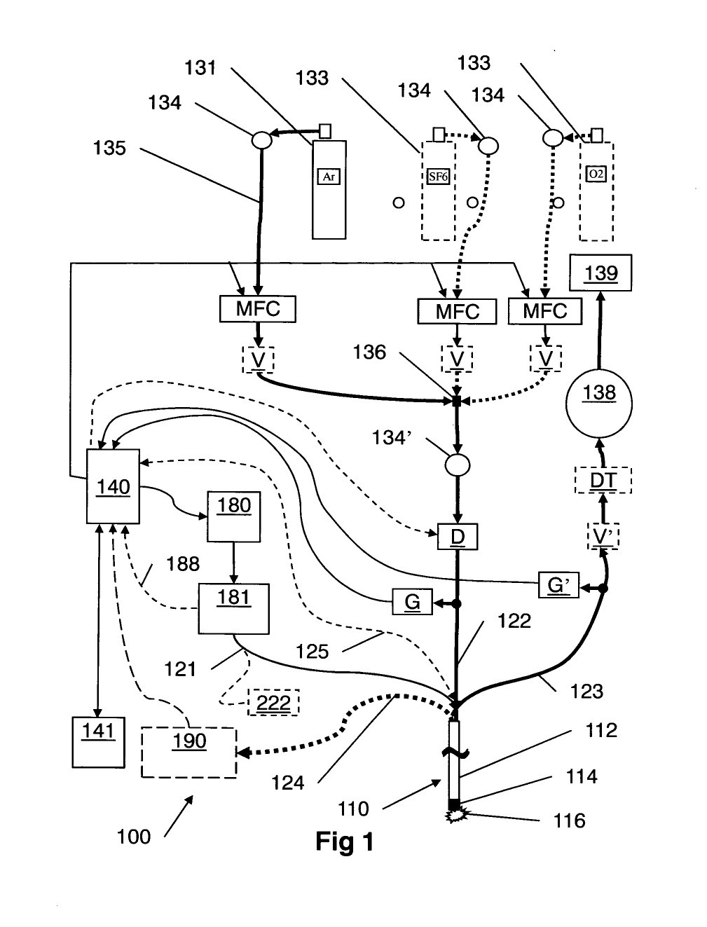

FIG. 1 schematically depicts a block diagram of a plasma system for treating CTO according to an exemplary embodiment of the current invention.

CTO treatment system 100 comprises a catheter 110 constructed and sized to be inserted into an occluded blood vessel, for example a coronary artery. Catheter 110 comprises an elongated flexible shaft 112 terminating at is distal end with a plasma head 114 for producing plasma 116 for ablating occlusions.

Shaft 112 is connected to at lest: RF input line 121; Gas input line 122; and Gas output line 123.

Optionally, Shaft 112 is further connected to optical fiber 124.

Optionally, Shaft 112 is further connected to electrical cable 125.

Gas delivery subs-system of CTO treatment system 100 comprises at least one gas tank 131. Gas tank 131 supplies gas used for plasma production. For example, gas tank 131 may contain compressed noble gas, for example Argon. Alternatively Helium gas may be used. Optionally mixture of few gases may be used.

Optionally a plurality of gas tanks are used, each containing different gas or gas mixture. In the exemplary embodiment of the invention, gas delivery sub-system comprises gas tanks 132 and 133 containing SF6 and O2 gas respectively.

Each gas tank is connected to a gas pressure regulator 134 for reducing gas pressure from the pressure in the tank to pressure of approximately 30 PSI.

Gas at reduced pressure flows in gas manifold lines 135 to optional Mass Flow Control (MFC). For clarity, only the manifold gas line connected to gas tank 131 is marked in this figure. Each MFC controls the gas flow in the respective manifold gas line 135 according to command signal received from control unit 140. Optionally, MFCs are used to control the ratio of different gases in the gas mixture delivered to catheter 110.

Optional directional one way valve V ensures that no back flow of gas will occur.

In embodiments having more than one gas tank, the gas flow from all manifold gas lines 135 is combined 136 and the pressure is reduced to approximately atmospheric pressure in regulator 134. Optionally, if only one gas tank is used, only one of regulators 134 and 134′ is used and directional valve V may be missing.

Demand valve D is for turning on and off the flow of gas, opening it only when low pressure is sensed in its distal end (close to the plasma head Demand valve D is optionally controlled by controller 140.

Optional pressure gauge G measures the gas pressure in input gas line 122 and optionally reports its reading to controller 140.

Spent gas from catheter 110 flows through output gas line 123. Optional pressure gauge G′ measures the gas pressure in output gas line 123 and optionally reports its reading to controller 140. Optional directional one way valve V′ prevents reverse gas flow in output gas line 123.

Optional drain tank DT may be used as a buffer before gas pump 138 which pumps spent gas out of output gas line 123. Optional drain tank DT may comprise a scrubbing filter for containing, absorbing or neutralizing bio-hazard, toxic, noxious or foul smelling elements which may be present in the spent gases. Spent gas is than vented to the atmosphere directly or through exhaust 139. Exhaust 139 may comprise scrubbing filter to eliminate contamination such as toxic gas or biologically hazardous particles.

Electrical power, for example RF power or pulsed DC power is generated in RF generator 180 which is preferably controlled by controller unit 140. RF power from RF generator 180 is coupled to the RF input line 121 through impedance matching circuit 181. Preferably, RF input line 121 is a coaxial electric cable. It should be noted that electrical energy transmitted in RF input line 121 may be in the form of RF or pulsed DC electrical signal.

According to an exemplary embodiment of the current invention RF frequency higher than 1 MHz is used. Preferably frequency of approximately 13 MHz is used, however lower or higher frequency may be used. According to an exemplary embodiment of the invention, RF power 0.3 to 8 Watt is used to allow etch rate of 1 to 10 mm/min, however higher or lower power levels and ablation rates may be used. Optionally the user may control the power level, the gas flow rate and the ablation rate.

Optionally, in some embodiments, processor 140 receives signal indicative of plasma process, for example by monitoring electrical plasma current or plasma impedance, for example through monitoring line 188. In some embodiments, impedance matching circuit 181 comprises a resistor and voltage developed on said resistor is indicative of plasma current. In some embodiments, said resistor is situated within the plasma head. In some embodiments, in close proximity to the plasma electrode.

Optional optical fiber 124 delivers optical signals generated by the plasma 116 and indicative of ablation products of said plasma to the optional optical spectrometer 190. Electrical signals from spectrometer 190 are reported to controller unit 140 and are used for analyzing the progress of the plasma ablation.

For example, optical spectrometer 190 may detect the abundance of phosphorus (P) in the living cells which does not exist in the plaque, for example by monitoring one of the phosphorus wavelengths, for example at 253 nm.

Additionally or alternatively, optical sensors (not seen in this figure) are installed within catheter 110 and are used for monitoring ablation progress. Optionally other types of sensors are used, for example impedance analysis for characterization of the head location and determining the vicinity to the artery wall and/or for determination of a device approaching the surface.

Said sensors receive power and report their reading to controller 140 through electric cable 125.

Controller unit 140 is preferably a computer such as a PC or a laptop computer. However, controller 140 may be a DSP or other data processing device. Controller 140 received user input and display user output through peripherals units 141 which may comprise some of: keyboard, mouse and/or other input devices, a display, printer and/or other output devices and optionally external storage devices and LAN or internet communication.

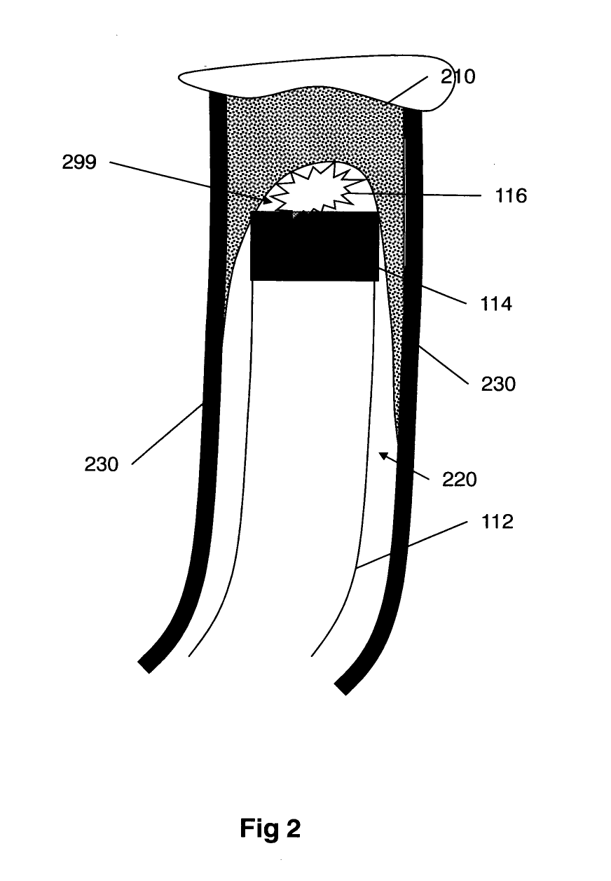

FIG. 2 schematically depicts the distal end of catheter 110 of a plasma system for treating CTO 100 inserted in a totally occluded blood vessel according to an exemplary embodiment of the current invention.

Distal end of catheter 110 comprises an elongated flexible shaft 112 terminating at is distal end with a plasma head 114 for producing plasma 116 for ablating occlusion 210 in blood vessel 220.

Preferably plasma head 114 is in close proximity or touching occlusion 210. Pump 138, which pumps the gas from space 299 in front of plasma head 114 through gas output line 123 in flexible shaft 112 of catheter 110 preferably creates low pressure between plasma head 114 and occlusion 210, keeping the plasma head in proximity to said occlusion, sucking the input gas trough the input tube 122 while opening the demand valve D due to the low pressure accumulated, and sucking out and removing the spent gas including ablation products. The created low pressure removes spent gas from the plasma space. Demand valve D preferably supply gas only when gas is being properly removed from the plasma space. Gas supply is interrupted, for example by automatic shutting off of demand valve D due to absence of low pressure in its distal end, if the gas return lumen is blocked, for example by blood or other debris. Optionally, when gas supply is interrupted, electric power to plasma electrode is also turned off.

Additionally or alternatively, the user pushes the catheter forwards until plasma head 114 makes contact with occlusion 210.

Optionally, steering mechanism, for example wires (not seen in this figure) within flexible shaft 112 direct plasma head so that plasma 116 ablate the occlusion 210 and spare the walls 230 of blood vessel 220.

Analyzing signals from spectrometer 190 or the sensors within plasma head 114, it may be possible to assess the progress of the occlusion ablation and direct the plasma head accordingly. Spectrometer 190 may be an optical spectrometer comprising a diffraction grating or a prism and a plurality of optical detectors such as pixilated 1D or 2D detector array. Alternatively, spectrometer 190 may comprise a scanning wavelength spectrometer or a plurality of sensor wherein at least few of said sensors are fitted with an optical filter tuned for a specific optical transmission band or bands.

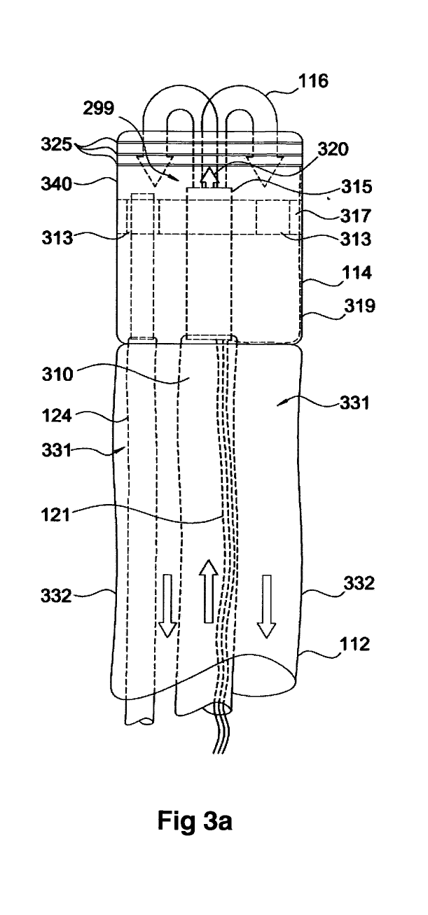

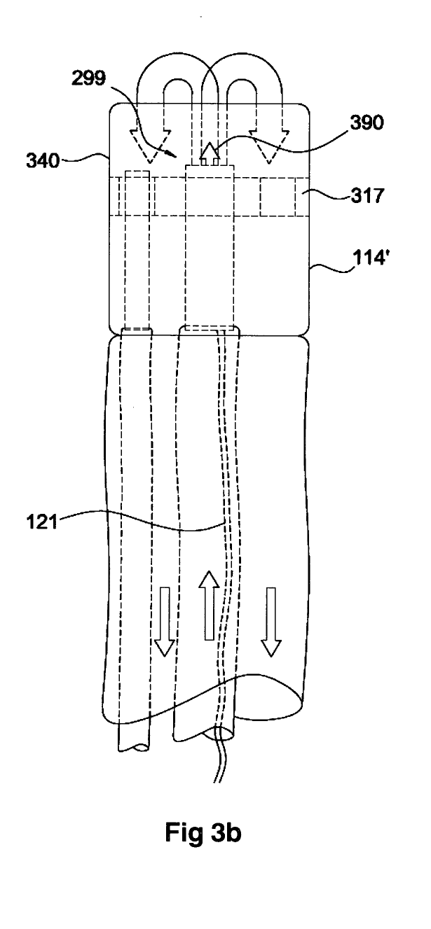

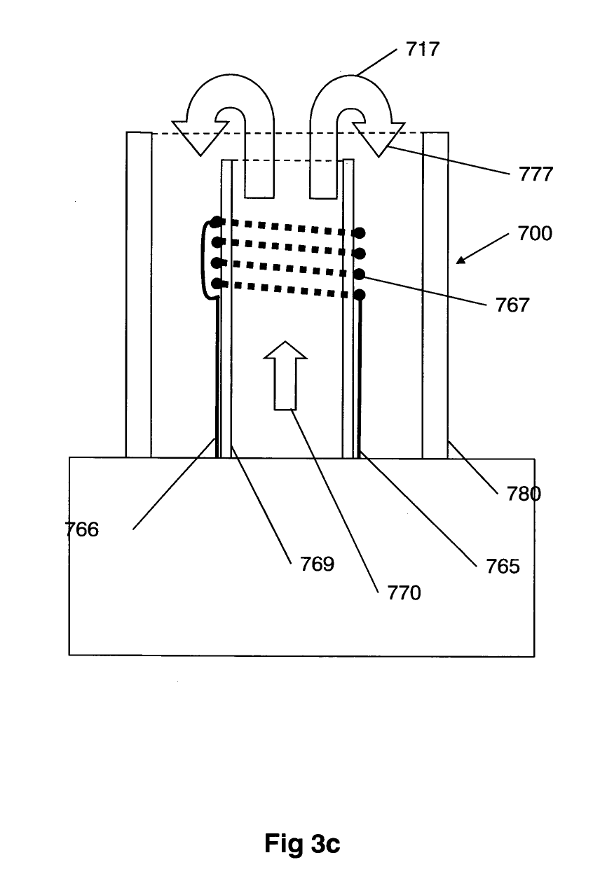

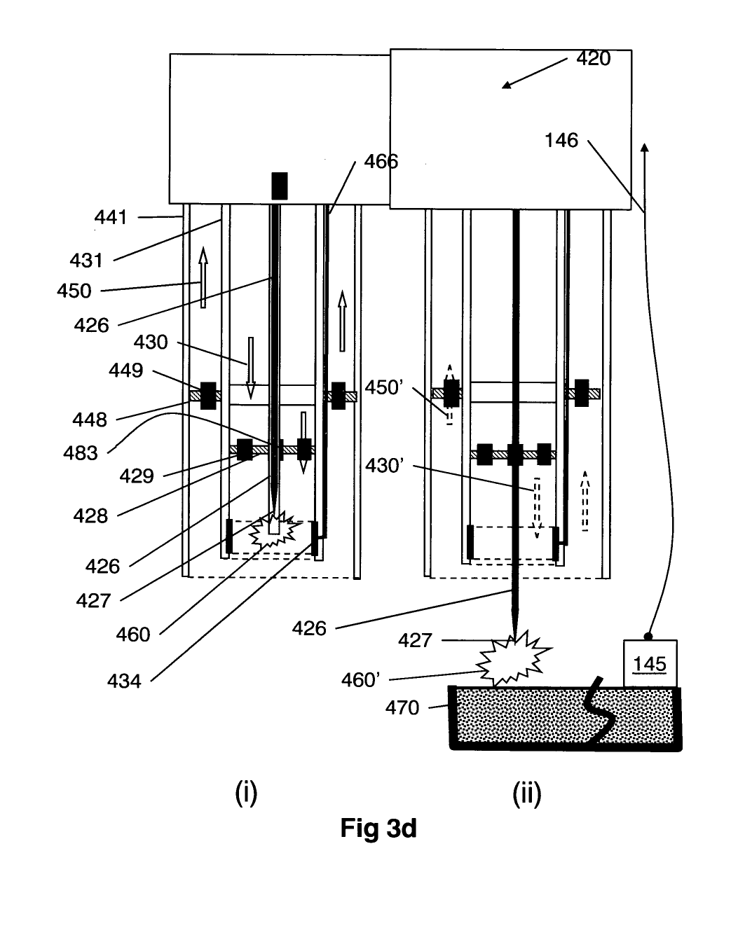

FIG. 3a schematically depicts distal end of a catheter showing some details of a bi-polar plasma head for CTO ablation according to an exemplary embodiment of the current invention.

Flexible shaft 112 preferably comprises a central gas input tube 310 which connects to gas input line 122. Gas use for plasma production flows through input tube 310 and past the central electrode 320.

RF energy transmitted from matching circuit 181 through coaxial electric wire 121 creates alternating electric potential between central electrode 320 and grounded RF electrodes 325 at inner side of the outer tube 340 of plasma head 114. Alternatively, grounded RF electrodes 325 are located at outer side of the outer tube 340 of plasma head 114. Alternatively, electrical grounding pad 222 (seen in FIG. 1) is attached to the patient and is used for the retune of plasma current.

Preferably central electrode is a sharp tip made of tungsten. Grounded RF electrodes are connected to the coaxial 121 with wire 319, optionally through a variable resistor. In some embodiments, electric return line is fitted with variable impedance device such as a variable resistor or resistor network, allowing to change the plasma production nature between mono-polar and bi-polar and combination of the like. Preferably, the variable impedance unit is located at or near the power supply, and is preferably controlled by the user.

Preferably, outer plasma tube 340 is made of quartz. Typical diameter of plasma head 114 is 1 mm and the length of outer tube 340 is 0.5 mm, however different sizes may be used within the scope of the current invention.

Plasma 116 ablate the occlusion in front of plasma head 114 and ablation products and spent gas are then pumped out by pump 138 through the output lumen 331 formed between input tube 310 and the inner surface of outer tube 332 of flexible shaft 112. Output lumen 331 is connected to gas output line 123.

Optical fiber 124 is preferably runs along the flexible shaft 112 within the output lumen 331. Optical fiber 124 collects optical signal produced by plasma 116 as it interacts with occlusion 210 and transmits the optical signals to spectrometer 190.

Base plate 317 secured to outer tube 332 of flexible shaft 112 carries central electrode 320 and outer plasma tube 340. Base plate 317 has a central gas input hole 315 for allowing gas flow from input tube 310 to the outer plasma tube, and a plurality of drain holes 313 for allowing spent gas and ablation product to flow from outer plasma tube 340 to output lumen 331.

Preferably, length of flexible shaft 112 is approximately 2 meters.

FIG. 3b schematically depicts distal end of a catheter showing some details of a mono-polar plasma head 114′ for CTO ablation according to an exemplary embodiment of the current invention.

Mono-polar plasma head 114′ depicted in FIG. 3b differs from bi-polar plasma head 114 of FIG. 3a by the absence of grounded RF electrodes 325 at the outer tube 340. According to the exemplary embodiment of the invention, central lead of coaxial RF input line 121 is connected to central electrode 390, while the outer shall of coaxial RF input line 121 is optionally left electrically unconnected at the distal end of the catheter.

Optionally, the outer shall of coaxial RF input line 121 is grounded and electrically connected to the patient body, preferably using a conductive pad (not seen in this figure) attached to the patient skin.

FIG. 3c schematically depicts a cross section of a plasma distal end of a catheter showing some details of inductively generated plasma head 700 for CTO ablation according to another exemplary embodiment of the current invention.

For simplicity, non essential details (some already depicted in other drawings) are not depicted in this figure. For simplicity, some parts that were already explained may not be marked in this figure.

In contrast to bi-polar plasma head 114 and mono-polar plasma head 114′, RF power supplies to inductively generated plasma head 700 is connected via lines 765 and 766 to a coil 767 wound around inner tube 769 which is carrying the gas flow 772. Coil 767 is preferably part of a tuned resonance circuit which may be a part of the impedance matching circuit. RF current in coil 767 excites the gas flow 770 in outer INNER tube 7 and thus creates plasma 717. In some embodiments, number or turns in coil 767 is limited, for example only few turns, and optionally as few as 1, 1.5 or 2 turns. Spent gas, together with ablation products return 777 through the lumen between inner tube 769 and outer tube 780.

For simplicity, the spacer that holds inner tube 769 to outer tube 780, which may be similar to Base plate 317 is not seen in this schematic drawing.

FIG. 3d(i) schematically depicts a cross section of a dual purpose plasma head 420 in bi-polar welding configuration, according to another exemplary embodiment of the current invention.

For simplicity, non essential details (some already depicted in other drawings) are not depicted in this figure. For simplicity, some parts that were already explained may not be marked in this figure.

Head 420 receives RF power from RF (optionally a coaxial) cable the hose. Preferably, the central conductor of the RF cable is connected to sliding central electrode 426 preferably ending with a sharp tip 427. central electrode 426 is kept substantially central to the inner tube 431, through which plasma gas flow 430 is provided by perforated separator 428 with at least one opening 429 for the gas flow.

In the depicted embodiment, separator 428 is fixed to the inner tube 431 and central electrode 426 may slide in the opening 483. However, other means for providing relative motion of central electrode 426 in respect to inner tube 431 may be used. For example separator 428 may be fixed to the central electrode and slide with it along the inner tube 431, or opening 483 and central electrode may have screwing grooves and central electrode may advance and retract by rotating the central electrode.

Bi-polar plasma 460 is produced between central electrode 483 and outer electrode 434 located on the inner proximal end of inner tube 431. In the exemplary embodiment, electrode 434 is connected to the RF circuit via conductor 466; however, the sections or the entire inner tube may be made conductive and used as a conductor.

Optionally, in this configuration and others, electrode 434 may be a coil such as coil 767, preferably located on the inside of the tube and act for dual plasma excitation—inductive and capacitive excitation.

Spent gas 450 is removed through the lumen created between inner tube 431 and outer tube 441. Preferably, inner tube is held central to outer tube by perforated separator 448 having at least one opening 449 for gas removal 450.

In some embodiments, the plasma is used for sterilization.

FIG. 3d(ii) schematically depicts a cross section of a dual purpose plasma head 420 in mono-polar ablation or coagulation configuration, according to another exemplary embodiment of the current.

For simplicity, non essential details (some already depicted in other drawings) are not depicted in this figure. For simplicity, some parts that were already explained may not be marked in this figure.

As depicted in this figure, central electrode 426 is pushed forward, using a mechanical or an electrical means (not seen in this figure), until its distal end 427 is outside outer tube 431. In this configuration, RF circuit is completed, creating mono-polar plasma 460′; via tissue 470 which is grounded by grounding pad 145 which is connected to the RF circuitry via grounding conductor 146. Preferably, RF power to annular grounding electrode 434 is turned off. However, central electrode 426 may be insulated along it length and exposed only at its tip 427. In this case, most of the current will flow through pad 145 even if annular electrode 434 is connected to the RF circuit.

In some embodiments, central electrode 426 makes contact with tissue 470 for performing coagulation or ablation without creating plasma.

In some embodiments, inner or outer tubes, or both may be made transparent, for example made of glass, quartz, clear plastic, sappier or other transparent material and be used as part of the light collecting and transporting system for transferring plasma emission light to light sensor such as spectrometer 190. Optionally, the tube is coated with light reflective material such as gold, Aluminum, silver or dielectric coating to maintain prevent light collected by the tube from escaping the tube, thus increasing light transmission to the sensor.

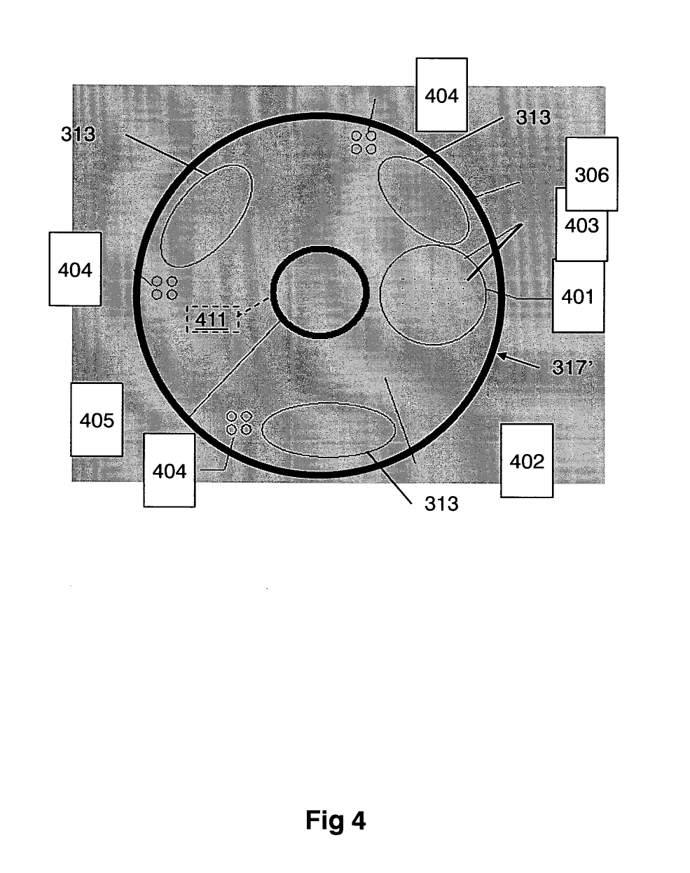

FIG. 4 schematically depicts a base plate having integrated sensors according to another aspect of the current invention.

Base plate with integrated sensors 317′ may replace base plate 317 as depicted in FIGS. 3a and FIG. 3b. In contrast to the embodiments depicted in FIGS. 3a and 3b, optional optical fiber 124 is missing. Consequently, spectrometer 190 depicted in FIG. 1 is also missing.

According to the exemplary embodiment depicted in FIG. 4, small sensor arrays 404 for local spectrum analysis perform plasma spatiel characterization. Optionally, readings of the local arrays, are used for steering the head to avoid undesired ablation of tissue as artery wall Each sensor (four are seen in this figure, but number of sensors in the array 404 may be larger or smaller) is covered by an optical filter having a predefined optical transmission characteristics.

Sensor arrays 404 and optional imaging sensor 401 receives electrical power and transmit signals to processor 140 via electrical cable 125. For example, an element or elements in sensor arrays 404 and optional imaging sensor 401 may be tuned to detect the abundance of phosphorus (P) in the living cells which does not exist in the plaque, for example by monitoring one of the phosphorus wavelength at 253 nm.

It should be noted that throughout this disclosure, the terms “light” and “optical” refers to electromagnetic radiation in the range from Ultra-Violet (UV) to the Infra-Red (IR) and is not limited to visible light. Preferably, transmission characteristics of said optical filters are selected that by comparing readings from the plurality of sensors, information regarding ablated tissue may be determined or estimated. For example, a determination of the biological subject being ablated may be determined by comparing known or experimentally investigated emission spectra of biological substrates such as: blood; blood clot, fatty occlusion; calcified occlusion; blood vessel wall; etc., with emission spectra detected by said sensor array 404 or spectrometer 190. Information resulting from said spectral analysis may be used for steering plasma head to ablate the occlusion and avoid the blood vessel walls. Similarly, Information resulting from said spectral analysis may be used for identifying the completion of the ablation of the occlusion and inform the user or automatically terminate the ablation process, for example by turning off RF generator 180.

In the embodiment of FIG. 4, the difference in detected emission in the plurality of sensor arrays 404 (three are depicted, but number of arrays may be larger or smaller), may enable the user to decide in which direction to direct the plasma head. Optionally processor 140 automatically analyzes electrical signals from the plurality of arrays 404 and recommend to the user in which direction to direct the ablation. Additionally or alternatively, processor 140 automatically directs the plasma ablation or halts the ablation.

Optional Image sensor 401 is preferably used for imaging at least part of the ablation field. Preferably, a lens (or an assembly of lenses) in front of image sensor 401 is used for focusing plasma induces emitted light onto the image sensor 401. Image sensor 401 is preferably a pixilated optical sensor array such as CCD array used in electronic cameras comprising a plurality of pixels 403. Optionally, image sensor 401 is covered with an optical filter. Optionally more than one image sensors 401 are used, each covered with an optical filter having different cartelistic transmission. Additionally or alternatively, plurality of image sensors 401 are used, each viewing different (optionally overlapping) field of view.

Base plate 317′ may be constructed from chip body 402 made of semiconducting solid state piece, for example silicon. Chip body 402 may be manufactured using electronics fabrication processes known in the art and integrate both electronic and structural elements of base plate 317′. Alternatively Base plate 317′ may be constructed from a structural part and have at least some of the electro-optical elements attached to the structural part.

FIG. 4 also shows lumen of inner tube 405 optionally used for gas input and RF electrode, and drain holes 313 used for returning of spent gas and ablation products to output lumen 331. FIG. 4 also shows outer plasma tube 340 which define the “chamber” of plasma confinement.

Optionally, in some embodiments, Base plate 317′ further comprises electrical circuit 411 receiving electrical power from input line 121 and conditioning it for plasma generation and transmitting conditioned electrical power to plasma electrode 405. Specifically, electrical circuit 411 may generate RF signal from DC signal. Additionally and optionally, circuit 411 may be part of impedance matching circuit or part of plasma current or impedance monitoring circuit.

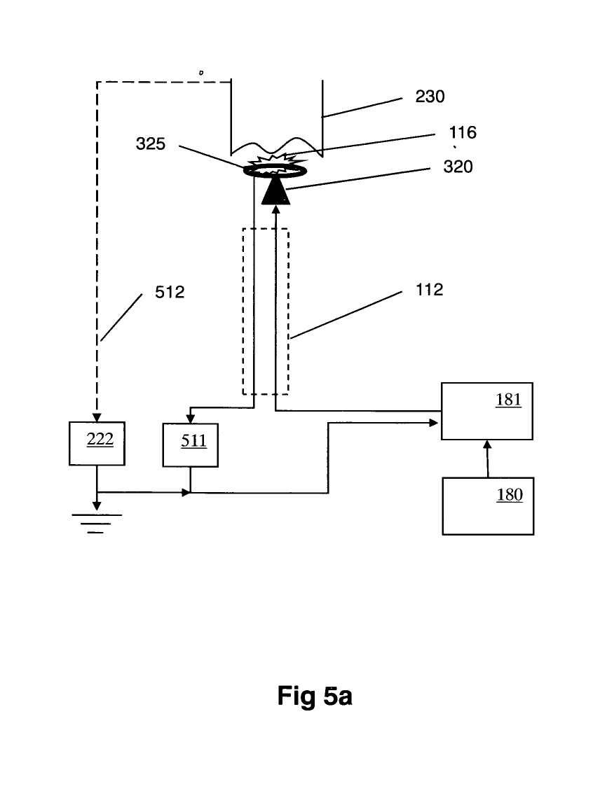

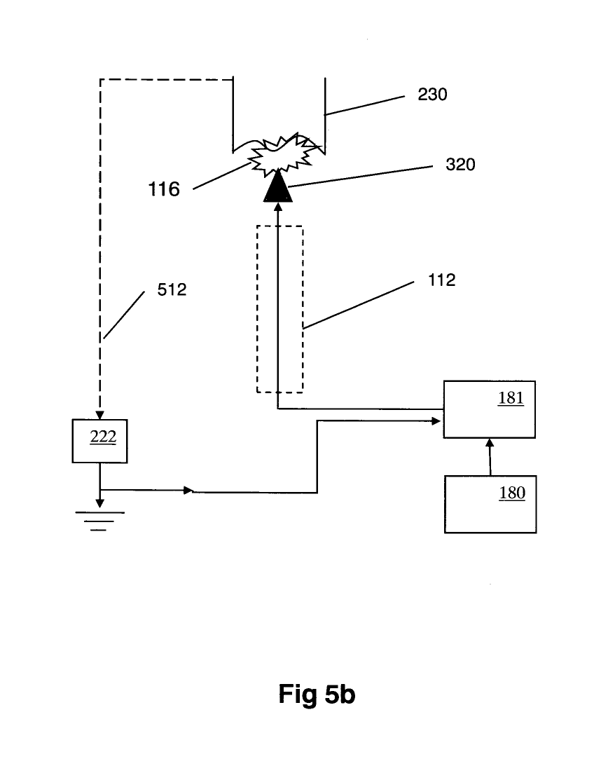

FIG. 5a schematically depicts the electrical connections of a bi-polar plasma system according to an exemplary embodiment of the current invention.

Optional variable impedance 511 is placed in the electrical return line.

When the impedance of variable impedance 511 is low, the electrical return current is flowing primarily through electrode 325 and variable impedance 511. Thus, the device acts as mainly bi-polar.

When the impedance of variable impedance 511 high, the electrical return current is flowing primarily through grounding electrode 222 electrically connected through the patient's body 512 to blood vessel's wall 230. Thus, the device acts as mainly mono-polar.

When the impedance of variable impedance 511 intermediate, the device acts as a combination of bi-polar and mono-polar.

FIG. 5b schematically depicts the electrical connections of a mono-polar plasma system according to an exemplary embodiment of the current invention. Electrical return current is flowing through grounding electrode 222 electrically connected through the patient's body 512 to blood vessel's wall 230. Thus, the device acts as mono-polar.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

It should be noted that any of the plasma heads, catheters, and other embodiments may be used within a body cavity as a part of a rigid or flexible endoscope. For example, the catheter may be inserted through the working channel of a colonoscope pry treating and removing polyps in the lower digestive tracks.

In some applications the plasma head according to embodiments of the current invention is used for generation of plasma for dental application. For example, sterilization of dental tissue or bleaching (Whitening) of tooth surface. In these embodiments, spent gas collection may be missing.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-39. (canceled)

40. A plasma head to be situated in proximity to body part to be treated, the plasma head comprising:
an input gas lumen configured to connect to a gas supply system capable of supplying gas to said input gas lumen;
an output gas lumen;
an outer tube defining plasma space between said outer tube and the body part to be treated, wherein input gas is capable of entering said plasma space through said input gas lumen, and spent gas is capable of exiting out of said plasma space through said output gas lumen; and
a plasma electrode capable of receiving electrical power from an RF generator via an electrical energy supply cable, and capable of generating plasma in said plasma space.

41. The plasma head of claim 40, wherein said input gas lumen and said output gas lumen are coaxial.

42. The plasma head of claim 41, wherein said input gas lumen is centrally to output gas lumen.

43. The plasma head of claim 42, wherein a wall of said input gas lumen is electrically conductive and is part of said electrical energy supply cable.

44. The plasma head of claim 40, further comprising an optical spectrometer, wherein signal from said spectrometer is capable of providing signal indicative of type of the treatment of the body part, and wherein said signal is used for controlling the treatment process.

45. The plasma head of claim 44, further comprising an optical fiber capable of transmitting light generated by plasma in said plasma space to said optical spectrometer, and wherein said optical spectrometer is situated outside the plasma head.

46. The plasma head of claim 44, wherein the spectrometer comprises a plurality of optical sensors each fitted with an optical filter having a predefined optical transmission.

47. The plasma head of claim 46, wherein at least one of said a plurality of optical filter having a predefined optical transmission is tuned to the emission wavelength of phosphorus.

48. The plasma head of claim 47, wherein said spectrometer is integrated in an electro-optical chip.

49. The plasma head of claim 48, further comprising an optical imager imaging the body part that is ablated.

50. The plasma head of claim 49, wherein said electro-optical chip define together with outer tube defining said plasma space between said outer tube and said body part.

51. The plasma head of claim 50, wherein said electro-optical chip further comprises said plasma electrode.

52. The plasma head of claim 40, wherein said outer tube further comprises grounding electrode on its inner surface, and wherein said grounding electrode is capable of being used for return plasma current from said plasma electrode.

53. The plasma head of claim 40, wherein said outer tube further comprises grounding electrode on its outer surface, and wherein said grounding electrode is capable of being used for return plasma current from said plasma electrode.

54. The plasma head of claim 40, further comprising a grounding electrode pad capable of being attached to the patient body, wherein said grounding electrode pad is capable of being used for retune plasma current from said plasma electrode.

55. The plasma head of claim 40, wherein grounded electrode is configured to be connected to ground through a variable resistor for plasma characteristic control.

56. The plasma head of claim 40, wherein said gas supply system is capable of supplying gas comprising gases selected from a group of: argon; helium; oxygen and SF6.

57. A method for plasma treatment of body tissue comprising the steps of:
supplying gas through an input gas lumen to a plasma head;
supplying electrical energy through electrical energy supply cable to the plasma head;
generating plasma in said gas using said electrical energy for treating parts of the body tissue; and
exhausting spent gas through an output gas lumen.

58. The method of claim 57, wherein said input gas lumen and said output gas lumen are coaxial.

59. The method of claim 57, further comprising:
spectrally analyzing light emitted as result of said plasma; and
using said spectral analysis for controlling treatment process.

60. The method of claim 57, further comprising:
spatially analyzing light emitted as result of said plasma; and
using said spatial analysis for aiming the treatment process.

61. A medical system for plasma treatment of body tissue comprising:
an elongated device having:
an input gas lumen;
an output gas lumen; and
electrical energy supply cable;
a gas supply system capable of supplying gas to said input gas lumen;
a gas pump capable of pumping spent gas from said output gas lumen;
a plasma head at a distal end of said elongated device to be situated in proximity to body part to be treated, said plasma head comprising:
an outer tube defining plasma space between said outer tube and the body part to be treated, wherein input gas enters said plasma space through said input gas lumen, and spent gas is exhausted out of said plasma space through said output gas lumen; and
at least a first plasma generating electrode capable of receiving electrical power from said electrical energy supply cable and generating plasma in said plasma space.

62. The medical system for plasma treatment of claim 61, wherein said first plasma generating electrode is a coil, inductively generating plasma.

63. The medical system for plasma treatment of claim 61, further comprising a second plasma generating electrode, and wherein plasma is generated as bi-polar plasma between said first and said second plasma generating electrodes.

64. The medical system for plasma treatment of claim 61, wherein said electrical power comprises a high frequency signal modulated at low frequency.
